(19) United States
(12) Patent Application Publication
(10) Pub. No.: US 2011/0052727 A1
(43) Pub. Date: Mar. 3, 2011

(54) Anti Influenza Nutritional Supplements

(76) Inventor: Hanan Polansky, Rochester, NY (US)

(21) Appl. No.: 12/550,525

(22) Filed: Aug. 31, 2009

(51) Int. Cl. A61K 33/04; A61K 36/00; A61K 36/82; A61K 36/54; A61K 36/484; A61K 31/704; A61K 31/353; A61K 31/192; A61K 31/11; A61P 31/16

(52) U.S. Cl. 424/702; 424/725; 424/729; 424/739; 424/757; 514/33; 514/456; 514/570; 514/701

(57) ABSTRACT

The invention presents nutritional supplements with anti influenza activities. In a preferred embodiment, the invention features administration to a subject an effective dose of a nutritional supplement that includes a combination of all or some of the following ingredients licorice, quercetin, green tea, cinnamon, propolis, and selenium.

### I. BACKGROUND OF THE INVENTION

According to the WHO website¹: “Seasonal influenza is an acute viral infection caused by an influenza virus. There are three types of seasonal influenza—A, B and C. Type A influenza viruses are further typed into subtypes according to different kinds and combinations of virus surface proteins. Among many subtypes of influenza A viruses, currently influenza A (H1N1) and A (H3N2) subtypes are circulating among humans. Influenza viruses circulate in every part of the world. Type C influenza cases occur much less frequently than A and B. That is why only influenza A and B viruses are included in seasonal influenza vaccines. Seasonal influenza is characterized by a sudden onset of high fever, cough (usually dry), headache, muscle and joint pain, severe malaise (feeling unwell), sore throat and runny nose. Most people recover from fever and other symptoms within a week without requiring medical attention. But influenza can cause severe illness or death in people at high risk. The time from infection to illness, known as the incubation period, is about two days. Seasonal influenza spreads easily and can sweep through schools, nursing homes or businesses and towns. When an infected person coughs, infected droplets get into the air and another person can breath them in and be exposed. The virus can also be spread by hands infected with the virus. Antiviral drugs for influenza are available in some countries and effectively prevent and treat the illness. There are two classes of such medicines, 1) adamantanes (amantadine and remantadine), and 2) inhibitors of influenza neuraminidase (oseltamivir and zanamivir). Some influenza viruses develop resistance to the antiviral medicines, limiting the effectiveness of treatment. Influenza epidemics occur yearly during autumn and winter in temperate regions. Illnesses result in hospitalizations and deaths mainly among high-risk groups (the very young, elderly or chronically ill). Worldwide, these annual epidemics result in about three to five million cases of severe illness, and about 250,000 to 500,000 deaths. Most deaths associated with influenza in industrialized countries occur among people age 65 or older. In some tropical countries, influenza viruses circulate throughout the year with one or two peaks during rainy seasons. Influenza can cause serious public health and economic problems. In developed countries, epidemics can result in high levels of worker absenteeism and productivity losses. In communities, clinics and hospitals can be overwhelmed when large numbers of sick people appear for treatment during peak illness periods. While most people recover from a bout of influenza, there are large numbers of people who need hospital treatment and many who die from the disease every year. Little is known about the effects of influenza epidemics in developing countries. The most effective way to prevent the disease or severe outcomes from the illness is vaccination. Safe and effective vaccines have been available and used for more than 60 years. Among healthy adults, influenza vaccine can prevent 70% to 90% of influenza-specific illness. Among the elderly, the vaccine reduces severe illnesses and complications by up to 60%, and deaths by 80%. Influenza vaccination is most effective when circulating viruses are well-matched with vaccine viruses.” However, influenza viruses are constantly changing, which reduces the effectiveness of vaccination. In some cases vaccination is also associated with serious side effects. A specific example is the changes of the H1N1 virus that causes the 2009 H1N1 pandemic. The limitations mentioned in the WHO's description of the current anti influenza methods, indicate the existence of a need for new anti influenza methods. The current invention presents such methods. ¹http://www.who.int/mediacentre/factsheets/fs211/en/index.html

### II. BRIEF SUMMARY OF THE INVENTION

In one aspect, the invention presents methods for preventing or treating an infection with an influenza virus. In a preferred embodiment, the methods feature administration to a subject an effective dose of a nutritional supplement (also called dietary supplement) that shows anti influenza activities. For example, to ameliorate a disease symptom resulting from a infection with an influenza virus, or to prevent the onset of such diseases, a nutritional supplement can be administered to the subject to reduce susceptibility to infection by an influenza virus, reduce viral infectivity, reduce viral absorption, reduce the cytopathic effects (CPE) of such infection, inhibit the virus growth or replication, reduce viral damage to human or animal tissue, reduce expression and/or secretion of inflammatory proteins or other inflammatory reactions, and reduce viral titers or yield.

### III. DETAILED DESCRIPTION OF THE INVENTION

In one aspect, the invention presents methods for preventing or treating an infection with an influenza virus. In a preferred embodiment, the methods feature administration to a subject an effective dose of a nutritional supplement with anti influenza virus activities.

A. Treatment protocols

1. Introduction

The following sections describe standard protocols for determining effective dose, and for agent formulation for use. Additional standard protocols and background information are available in books, such as In vitro Toxicity Testing Protocols (Methods in Molecular Medicine, 43), edited by Sheila O'Hare and C K Atterwill, Humana Press, 1995; Current Protocols in Pharmacology, edited by: S J Enna, Michael Williams, John W Ferkany, Terry Kenakin, Roger D Porsolt, James P Sullivan; Current Protocols in Toxicology, edited by: Mahin Maines (Editor-in-Chief), Lucio G Costa, Donald J Reed, Shigeru Sassa, I Glenn Sipes; Remington: The Science and Practice of Pharmacy, edited by Alfonso R Gennaro, 20^{th} edition, Lippincott, Williams & Wilkins Publishers, 2000; Pharmaceutical Dosage Forms and Drug Delivery Systems, by Howard C Ansel, Loyd V Allen, Nicholas G Popovich, 7^{th} edition, Lippincott Williams & Wilkins Publishers, 1999; Pharmaceutical Calculations, by Mitchell J Stoklosa, Howard C Ansel, 10^{th} edition, Lippincott, Williams & Wilkins Publishers, 1996; Applied Biopharmaceutics and Pharmacokinetics, by Leon Shargel, Andrew B C Yu, 4^{th} edition, McGraw-Hill Professional Publishing, 1999; Oral Drug Absorption: Prediction and Assessment (Drugs and the Pharmaceutical Sciences, Vol 106), edited by Jennifer B Dressman, Hans Lennernas, Marcel Dekker, 2000; Goodman & Gilman's The Pharmacological Basis of Therapeutics, edited by Joel G Hardman, Lee E Limbird, 10^{th} edition, McGraw-Hill Professional Publishing, 2001.

2. Effective dose

Compounds can be administered to a subject, at a therapeutically effective dose, to treat, ameliorate, or prevent a disease. Monitoring of patient status, using either systemic means, standard clinical laboratory assays, or assays specifically designed to monitor the bioactivity of such compounds on the influenza virus and on its effects, can be used to establish the effective therapeutic dose and to monitor this effectiveness.

Prior to patient administration, techniques standard in the art may be used with any agent described herein to determine the LD₅₀ and ED₅₀ (lethal dose which kills one half the treated population, and effective dose in one half the population, respectively) either in cultured cells or laboratory animals. The ratio LD₅₀/ED₅₀ represents the therapeutic index which indicates the ratio between toxic and therapeutic effects. Compounds with a relatively large index are preferred. These values are also used to determine the initial therapeutic dose.

3. Formulation for use

Those skilled in the art recognize a host of standard formulations for the agents described in this invention. For instance, the agent may be given orally by delivery in a tablet, capsule or liquid syrup. Those skilled in the art recognize pharmaceutical binding agents and carriers which protect the agent from degradation in the digestive system and facilitate uptake. Similarly, coatings for the tablet or capsule may be used to ease ingestion thereby encouraging patient compliance. If delivered in liquid suspension, additives may be included which keep the agent suspended, such as sorbitol syrup and the emulsifying agent lecithin, among others, lipophilic additives may be included, such as oily esters, or preservatives may be used to increase shelf life of the agent. Patient compliance may be further enhanced by the addition of flavors, coloring agents or sweeteners. In a related embodiment the agent may be provided in lyophilized form for reconstitution by the patient or his or her caregiver.

The agents described herein may also be delivered via buccal absorption in lozenge form. Similarly, compounds may be included in the formulation which facilitate transepithelial uptake of the agent. These include, among others, bile salts and detergents.

In every case, therapeutic agents destined for administration outside of a clinical setting may be packaged in any suitable way that assures patient compliance with regard to dose and frequency of administration.

4. Clinical Trials

Another aspect of current invention involves monitoring the effect of an agent on a treated subject in a clinical trial. For example, to study the effect of a test agent in a clinical trial, blood may be collected from a subject before, and at different times following treatment with such an agent. The copy number of a influenza virus may be assayed in the blood, or the levels of expression of an affected gene, may be assayed by, for instance, Northern blot analysis, or RT-PCR, known in the art, or by measuring the concentration of the protein by one of the methods known in the art, or measuring the effect on affected cells using some method known in the art. In this way, the copy number, or expression profile of a gene of interest or its mRNA, or the morphology or behavior of certain cells, may serve a surrogate or direct biomarker of treatment efficacy. Accordingly, the response may be determined prior to, and at various times following agent administration. The effects of any therapeutic agent of this invention may be similarly studied if, prior to the study, a suitable surrogate or direct biomarker of efficacy, which is readily assayable, was identified.

### IV. EXAMPLES

Many nutritional agents have been identified for their antiviral activities and have been reported to inhibit infectivity and replication of a broad spectrum of viruses, among them, the influenza virus. Large number candidate substances and their anti viral functions have been identified by a combination of in vitro and in vivo studies using different biological assays. However, no claims have been made on the capacity of the combination of these substances to reduce infectivity and replication of the influenza virus. Furthermore, no claims have been made on the capacity of the combination of these substances to decrease the viral load, and as a result, decrease the risk of developing a disease, or decrease the severity of a current disease, which is developing or was developed following the viral infection. The following section will show examples of nutritional agents, witch constitute the compound of the nutritional supplement GENE-EDEN, including Glycyrrhizic acid/Glycyrrhizin, Quercetin, Epigallocatechin gallate, Cinnamon, Selenium and Propolis, with such effects.

A. Quercetin

Quercetin is a flavonoid and, or more specifically, a flavonol. It is the aglycone form of a number of other flavonoid glycosides, such as rutin and quercitrin, found in citrus fruit, buckwheat and onions. Quercetin forms the glycosides quercitrin and rutin together with rhamnose and rutinose, respectively. Quercetin is classified as IARC group 3 (no evidence of carcinogenicity in humans).

1. Influenza A Virus (Strain A/Puerto Rico/8/1934 H1N1), ICR Mice, Reduces Susceptibility to Infection

An in vivo experiment² showed that stressful exercise increases mortality from the influenza virus by 24% and that treatment with quercetin offsets the increase in mortality associated the exercise. Moreover, the experiment also showed that treatment with quercetin decreases the mortality in the control group (infected and did not exercise) by 22%. This experiment examined the effects of quercetin feedings on susceptibility to the influenza virus A/Puerto Rico/8/34 (H1N1) following stressful exercise. The experiment randomly assigned mice to one of four treatment groups: exercise-placebo, exercise-quercetin, control-placebo, or control-quercetin. Exercise consisted of a run to fatigue (approximately 140 min) on a treadmill for 3 consecutive days. The experiment administered quercetin (12.5 mg/kg) via gavage for 7 days before viral challenge. At 30 min after the last bout of exercise or rest, mice (n=23-30) were intranasally inoculated with a standardized dose of influenza virus (0.04 hemagglutinating units). Mice were monitored daily for morbidity (time to sickness), symptom severity, and mortality (time to death) for 21 days. Exercise stress was associated with an increased susceptibility to infection [morbidity, mortality, and symptom severity on days 5-7 (P<0.05)]. Quercetin offset the increase in susceptibility to infection [morbidity, mortality, and symptom severity on days 5-7 (P<0.05)] that was associated with stressful exercise. ² Davis J M, Murphy E A, McClellan J L, Carmichael M D, Gangemi J D. Quercetin reduces susceptibility to influenza infection following stressful exercise. Am J Physiol Regul Integr Comp Physiol. August 2008;295(2):R505-9.

2. Influenza A Virus (Strain A/Wilson-Smith/1933 H1N1), MDCD cells, reducing Cytopathic Effect (CPE)

An in vitro experiment³ showed that quercetin 3-rhamnoside (Q3R) possesses strong anti-influenza A/WS/33 virus activity, reducing the formation of a visible cytopathic effect (CPE). Q3R also inhibited virus replication in the initial stage of the infection by indirect interaction with virus particles. This experiment investigated the antiviral activity of Q3R from Houttuynia cordata against the influenza A/WS/33 virus using a CPE reduction method. Madin-Darby canine kidney (MDCK) cells were infected with pretreated or untreated (with 10/100 μg/ml Q3R) influenza A/WS/33 virus for 1 h at 37° C. The experiment observed the morphology of cells determined the antiviral activity after 2 days of incubation. The assay results demonstrated that Q3R possessed strong antiviral activity of about 86% against influenza A/WS/33 virus at concentration of 100 μg/ml, and antiviral activity of about 66% at the same virus at concentration of 10 μg/ml. Oseltamivir also did show moderate antiviral activity of about 58% against influenza A/WS/33 virus at concentration of 100 μg/ml, and weak antiviral activity of less than 49% at concentrations of less than 10 μg/ml. Q3R and Oseltamivir were not toxic to MDCD cells with cell viability of about 100% at concentration of 100 μg/ml. ³ Choi H J, Song J H, Park K S, Kwon D H. Inhibitory effects of quercetin 3-rhamnoside on influenza A virus replication. European Journal of Pharmaceutical Sciences, Online Mar. 14, 2009.

3. Influenza A Virus (Strain A/Leningrad/1/1954 H1N1), Chicken Embryos, Inhibition of Virus Growth

An in vivo experiment⁴ showed that KV-8, a preparation composed from 89% quercetin mixture, causes a profound inhibition of influenza A virus growth. In this experiment, culture of influenza A virus, strain A/Leningrad/54/1, was grown in the allantois cavities of 9-10 day chicken embryos. The test preparations (KV-8 at concentrations of 2.5, 0.25, 0.025 and 0.0025%) and the virus were simultaneously introduced into the embryos. The results showed that KV-8 in a concentration interval of 0.25-2.5% inhibited the growth of human influenza virus A/Leningrad/54/1 by 100%, and suppressed the replication process by 60% in a concentration interval of 0.0025-0.025%.

4. Influenza A Virus (Strain A/Udorn/317/1972 H3N2), Mice, Reduction of Viral Induced Lung Damage and Oxidative Stress ⁴É. Berezin, A. P. Bogoyavlenskii, V. P. Tolmacheva, D. Yu. Korul'kin, S. S. Khudyakova, S. V. Levandovskaya. Antiviral Activity of Preparations from Herbs of the Crassulaceae Family. Pharmaceutical Chemistry Journal. October 2002;36(10): 546-547.

An in vivo experiment⁵ showed that influenza virus infection in mice was associated with marked changes in lung morphology (epithelial damage and infiltration of leukocytes) and development of oxidative stress (increased superoxide radical production and lipid peroxidation), and that supplementation of quercetin, given orally, resulted in a significant decrease of these symptoms. In this experiment, BALB/c male mice were infected intranasally with influenza virus A/Udorn/317/72(H3N2) and supplemented orally with quercetin in a dose of 1 mg/day for 5 consecutive days. Results showed that oral supplementation of quercetin reduced the severity of infection. The epithelial damage and leukocytic influx were significantly reduced. Superoxide radicals production in influenza-infected mice were increased 1.5-2 fold compared to the normal control. Supplementation with quercetin significantly reduced their levels. Lipid peroxidation (LPO) products levels were raised by 85% in the influenza group compared to the normal control group. A significant (P<0.05) decrease of LPO to 43% was observed after supplementation with quercetin. ⁵ Kumar P, Sharma S, Khanna M, Raj H G. Effect of Quercetin on lipid peroxidation and changes in lung morphology in experimental influenza virus infection. Int J Exp Pathol. June 2003;84(3):127-33.

B. Epigallocatechin Gallate (EGCG)

Epigallocatechin gallate (EGCG), also known as Epigallocatechin 3-gallate, is a type of catechin and is the most abundant catechin in green tea. It is the ester of epigallocatechol and gallic acid.

1. Influenza A Virus (Strain A/Chile/1/83 H1N1), MDCK Cells, Inhibition of Influenza Virus Replication

An in vitro experiments⁶ showed that epigallocatechin gallate (EGCG) inhibits influenza virus replication in cell culture and causes direct virucidal effect. In this experiment, polyphenolic compound catechins ((−)-epigallocatechin gallate (EGCG), (−)-epicatechin gallate (ECG) and (−)-epigallocatechin (EGC)) from green tea were evaluated for their ability to inhibit influenza virus replication in cell culture and for potentially direct virucidal effect. Madin-Darby canine kidney (MDCK) cells were infected with influenza A/Chile/1/83 (H1N1) virus and than treated with catechins at different concentration. After incubation time that ranged between 8 hours to 3 days, cells were evaluated for antiviral effect by plaque inhibition assay, virus growth inhibition assay, hemagglutination inhibition assay, quantitative RT-PCR analysis and neuraminidase inhibition assay. Among the test compounds, EGCG was found to be the most potent inhibitor of influenza virus replication in MDCK cell culture. The 50% effective inhibition concentration (EC50) of EGCG for influenza A virus was 22-28 μM. EGCG exhibited the most effective hemagglutination inhibition activity. Quantitative RT-PCR analysis revealed that, at high concentration, EGCG also suppressed viral RNA synthesis in MDCK cells (about 80% inhibition at 500 μM). EGCG inhibited the neuraminidase activity more effectively than the other catechins. Reduction of half enzymatic activity was shown at relatively high concentration, about 350 μM for EGCG. Evaluation of cellular toxicity of catechins showed that the estimated dose of EGCG that reduced cell viability about 50% (CC50) was 275.4±22.8 μM. ⁶ Song J M, Lee K H, Seong B L. Antiviral effect of catechins in green tea on influenza virus. Antiviral Res. November 2005;68(2):66-74.

2. Influenza A Virus (Strain A/Puerto Rico/8/1934 H1N1), MDCK Cells, Inhibition of Virus Infection

An in vitro experiment⁷ showed that -epigallocatechin-3-O-gallate (EGCG) posses anti-influenza A virus activity and that a series of fatty acid monoester derivatives of EGCG (containing long alkyl chains) can enhance the antiviral activity. In this experiment, the anti-influenza A/PR8/34 (H1N1) virus protective effects of EGCG and its derivatives (addition of straight-chain fatty acids to the phenolic hydroxyl groups of EGCG) was evaluated. A monolayer of Madin-Darby canine kidney (MDCK) cells was transfected with EGCG and its derivatives 2 hours prior to infection with the virus. The cell monolayer was rinsed to remove remaining EGCG derivative in the cell culture medium, then the virus was introduced. The antiviral activities of each sample were assessed by the plaque formation assay. All compounds inhibited virus infection in a dose dependent manner. The EC50 values showed that the antiviral activities of EGCG-monoesters were enhanced in an alkyl chain length-dependent manner. In particular, the EC50 of EGCG-C16 was approximately 4 μM and its inhibitory effect was 24-fold higher than EGCG. This remarkable enhancement in antiviral activity can be attributed to the high efficiency of cellar uptake of EGCG-C16 as a result of its improved cell membrane permeability. EGCG was less toxic than most of its derivatives. ⁷ Mori S, Miyake S, Kobe T, Nakaya T, Fuller S D, Kato N, Kaihatsu K. Enhanced anti-influenza A virus activity of (−)-epigallocatechin-3-O-gallate fatty acid monoester derivatives: effect of alkyl chain length. Bioorg Med Chem Lett. Jul. 15, 2008;18(14):4249-52.

3. Influenza A Virus (Strain A/Puerto Rico/8/1934 H1N1), RBC Cells, Inhibition of Virus Absorption

In vitro and in ovo experiments⁸ showed that epigallocatechin (EGC) and its derivatives with different alkyl chain length exert pronounced inhibitory effects for all six influenza subtypes tested including three major types of currently circulating human influenza viruses (A/H1N1, A/H3N2 and B type), H2N2 and H9N2 avian influenza virus. The compounds strongly inhibit adsorption of the viruses on red blood cell (RBC). They also restrict the growth of avian influenza virus as it was seen in an in ovo experiment. In this experiment, hemagglutination inhibition (HI) assay was employed to evaluate the effects of the catechin derivatives on viral adsorption to target cells. Catechin solutions (25 μl) in serial two-fold dilutions in PBS were mixed with an equal volume of influenza virus solution (500 HAU/25 μl). After a 1 hour incubation at room temperature, 50 μl of the solution was mixed with an equal volume of a 1% chicken erythrocyte suspension and incubated for 30 min at room temperature. All of the tested compounds exhibited complete inhibition of viral adsorption onto RBCs in a concentration range of 20-120 μM, depending on the virus type tested. An in ovo experiment was conducted with 10 fertilized eggs (11-dayold). The eggs were inoculated with avian influenza A/Chicken/Korea/ms96/96 (H9N2) virus suspension containing 10-fold of egg infectious dose 50% (EID50)/50 μl mixed with various concentrations of catechin derivatives (50 μl). After 3 days, allantoic fluids were harvested and titrated by hemagglutination (HA) assay. Results revealed a pronounced inhibition in virus propagation at concentration ranged from 5.1 to 10.1 μM. ⁸ Song J M, Park K D, Lee K H, Byun Y H, Park J H, Kim S H, Kim J H, Seong B L. Biological evaluation of anti-influenza viral activity of semi-synthetic catechin derivatives. Antiviral Res. November 2007;76(2):178-85.

4. Influenza A Virus (Strain A/Yamagata/120/86 H1N1), MDCK Cells, CRBC Cells, Inhibition of Virus Infectivity

An in vitro experiment⁹ showed that Epigallocatechin Gallate (EGCG) blocks the infectivity of influenza A virus by inhibit its adsorption to the cells. In this experiment, Madin-Darby canine kidney (MDCK) cells were inoculated with a mixture of approximately 200 pfu virus and EGCG, allowing 60 min for virus adsorption. Plaques were than counted, and the percentage of plaque inhibition was calculated. EGCG strongly inhibited the infectivity of influenza virus. Even concentrations as low as 1.5 μM ECCG inhibited almost 100% of the plaque forming activity of the viruses after 60 min treatment. Short-time contact (5 minutes) of EGCG with the virus also effectively inhibited the infectivity. Furthermore, the experiment examined whether EGCG is effective if added after adsorption of virus to MDCK cells. Influenza A viruses were exposed to the cells for 30 minutes. EGCG was than added to virus-adsorbed cells for 15 minutes. Although the effective concentration of EGCG was higher, the addition of EGCG post viral absorption inhibited the plaque formation. Next, 25 μl of influenza A virus suspension were mixed with an equal volume of EGCG and maintained for 5 or 60 minutes at room temperature. 50 μl of the original solution and all dilutions of the mixture were than incubated with an equal volume of 0.5% chicken erythrocyte (CRBC) suspension for 60 minutes at room temperature for haemagglutination. Observation by electron microscopy showed that EGCG agglutinated virus particles and prevented the absorbance of the virus to cells. ⁹Nakayama M, Suzuki K, Toda M, Okubo S, Hara Y, Shimamura T. Inhibition of the infectivity of influenza virus by tea polyphenols. Antiviral Res. August 1993;21(4):289-99.

C. Cinnamaldehyde or Cinnamic Acid

Cinnamic aldehyde or cinnamaldehyde (more precisely trans-cinnamaldehyde) is the chemical compound that gives cinnamon its flavor and odor. Cinnamaldehyde occurs naturally in the bark of cinnamon trees and other species of the genus Cinnamomum like camphor and cassia. These trees are the natural source of cinnamon, and the essential oil of cinnamon bark is about 90% cinnamaldehyde. Most cinnamaldehyde is excreted in urine as cinnamic acid, an oxidized form of cinnamaldehyde.

1. Influenza A Virus (Strain A/Puerto Rico/8/1934 H1N1), MDCK Cells, Inhibition of Virus Growth

In vitro and in vivo experiments¹⁰ showed that trans-cinnamaldehyde (CA), a compound found in the bark of cinnamon trees, posses an inhibitory effect on the growth of influenza A/PR/8 virus. In these experiments, a one hour drug treatment was initiated at various times post viral infection in Madin-Darby canine kidney (MDCK) cells, using a fixed dose of CA (40 μM), and than, under the same treatment schedule, the cells were treated with various concentrations of CA (20-200 μM). The results showed a maximum inhibitory effect (29.7% virus yield of control) when CA treatment (40 μM) was given 3 hours post infection. Treatment with various concentrations (20-200 μM) inhibited the virus growth in a dose-dependent manner, and, at 200 μM, the virus yield was reduced to an undetectable level. Analyses showed that CA inhibited viral protein synthesis at the post-transcriptional level. In mice infected with the lung-adapted PR-8 virus, inhalation (50 mg/cage/day) and nasal inoculation (250 μg/mouse/day) of CA significantly increased survival rates on the 8 days to 100% and 70%, respectively, in contrast to a survival rate of 20% in untreated controls. Importantly, inhalation of CA caused virus yield reduction by 1 log in bronchoalveolar lavage fluid on day 6 after infection, compared with that of untreated controls. ¹⁰ Hayashi K, Imanishi N, Kashiwayama Y, Kawano A, Terasawa K, Shimada Y, Ochiai H. Inhibitory effect of cinnamaldehyde, derived from Cinnamomi cortex, on the growth of influenza A/PR/8 virus in vitro and in vivo. Antiviral Res. April 2007;74(1): 1-8.

2. Influenza A Virus (Strain A/Puerto Rico/8/1934 H1N1), Mice, Suppression of Interleukin-1 Alpha and Fever Production

An in vivo experiment¹¹ showed that treatment with extracts of Cinnamomum Cassia, a type of cinnamon, reduces fever and illness in mice infected with influenza virus. In this experiment, female mice were intranasally infected or mock infected with 2000-3000 plaque forming units of influenza virus A/PR/8/34 H1N1. Hot water extracts of Cinnamon (C. cassia) and other plants were orally applied by gavage to the mice 3 times daily for 4 days starting a day before infection. Cinnamon was further fractionated by sequential extractions with organic solvents to evaluate the properties of its compounds. The results showed that cinnamon significantly suppressed the influenza induced interleukin-1 alpha (induce fever) and fever production in infected mice compared with infected water administration mice. Cinnamon suppression was the strongest among the tested herbs. Cinnamon fractions extracted significantly reduced rectal temperatures and fever production in infected mice compared to control mice. ¹¹ Kurokawa M, Kumeda C A, Yamamura J, Kamiyama T, Shiraki K. Antipyretic activity of cinnamyl derivatives and related compounds in influenza virus-infected mice. Eur J Pharmacol. May 1, 1998;348(1):45-51.

D. Glycyrhhizic Acid

The flavoring agent licorice is derived from the root of Glycyrrhiza glabra, Glycyrrhiza uralensis or Glycyrrhiza inflate. The licorice root contains glycyrrhizic acid (GA), also called glycyrrhizin, or glycyrrhizinic acid.

1. Influenza A Virus (Strain A/Puerto Rico/8/1934 H1N1), A549 Cells, Inhibition of RANTES Secretion

An in vitro experiment¹² showed that Glycyrrhiza uralensis (Licorice) possesses a strong inhibitory effect on the secretion of RANTES (a chemotactic cytokines) induced by the infection of cells with influenza A virus. In this experiment, human bronchial epithelial cells (A549) were inoculated with H1N1 influenza A virus. After an absorption period of 1 hour, the cells were incubated in medium with the absence or presence of licorice extract. The results showed that influenza A virus H1N1 infection evoked a markedly stimulation of RANTES production from basal 6±3 to 1876±55 μg/ml after 72 hours inoculation of A549 cells. In comparison, RANTES concentration increased slightly to 49±8 pg/ml after 72 h incubation in the absence of H1N1 virus. An exposure of H1N1-infected A549 epithelial cells to 20, 100 and 200 μg/ml of licorice extract inhibited RANTES secretion by 24.5, 66.3 and 97.0 percents respectively. IC(50) values ranged from 35 to 48 μg/ml. Furthermore, cytotoxicity measurement results ruled out direct toxicity of licorice on A549 cells as a possible explanation for its inhibitory effect on RANTES release. To summarize, Glycyrrhiza uralensis (Licorice) extract markedly suppressed influenza A virus-induced RANTES secretion by human bronchial epithelial cells, suggesting that this extract may be beneficial for the treatment of chronic inflammatory conditions followed by viral infection. ¹² Ko H C, Wei B L, Chiou W F. The effect of medicinal plants used in Chinese folk medicine on RANTES secretion by virus-infected human epithelial cells. J Ethnopharmacol. Sep. 19, 2006;107(2):205-10.

2. Influenza A Virus (Strain A/Kumamoto/1/1965 H2N2), Mice, Increase in Mean Survival Time, Reduction of Virus Titers

An in vivo experiment¹³ showed that Glycyrrhizin treatment reduces morbidity and mortality of mice infected with lethal doses of influenza virus. In this experiment, mice were infected with the influenza A/Kumamoto/1/65 virus by inhalation of 20 ml of the virus solution. Mice exposed to the influenza virus were treated with various doses (1.25 to 80 mg/kg of body weight) of Glycyrrhizin 1 day before infection and 1 and 4 days post infection. Infected mice treated with saline (0.2 ml/mouse) served as controls. The antiviral effects of Glycyrrhizin were evaluated on the basis of survival rate, mean survival time in days, virus growth in lung tissues, and lung consolidation scores. The results showed that all of the mice that had been exposed to 10-50% lethal doses of the virus, and that were treated with Glycyrrhizin (10 mg/kg) 1 day before infection and 1 and 4 days post infection, survived over the 21-day experimental period. At the end of this period, the mean survival time (in days) for the control mice treated with saline was 10.5 days, and there were no survivors. The virus titers in the lungs of the treated group of mice 2 to 6 days after the infection were more than 10 times lower of those in the lungs of the control mice. On day 6, viral activity was not detected in the lungs of the treated mice, while the lungs of the control mice had titers that remained very high (5×10⁷ EID50 s/ml). ¹³ Utsunomiya T, Kobayashi M, Pollard R B, Suzuki F. Glycyrrhizin, an active component of licorice roots, reduces morbidity and mortality of mice infected with lethal doses of influenza virus. Antimicrob Agents Chemother. March 1997;41(3):551-6.

E. Selenium

Selenium is a chemical element with the atomic number 34, represented by the chemical symbol Se, and an atomic mass of 78.96. Selenium is a semi metal that rarely occurs in its elemental state in nature. It is toxic in large amounts, but trace amounts of it are necessary for normal cellular function in most, if not all, animals, forming the active center of the enzymes glutathione peroxidase and thioredoxin reductase and three known deiodinase enzymes.

1. Influenza A Virus (Strain A/Wilson-Smith/1933 H1N1), MDCK Cells, Inhibition of Virus Yield

An in vitro experiment¹⁴ showed that selenazofurin, an organoselenium compound, is a potent inhibitor of viral replication in cells infected with influenza A virus. In this experiment, Madin Darby canine kidney (MDCK) cells were treated with seven concentrations (1,000, 320, 100, 32, 10, 3.2, 1.0, ug/ml) of selenazofurin 15 minutes before virus exposure. The cells were then exposed to Influenza A/NWS/33 (H1N1) virus inoculum of 100 cell culture infectious doses. Viral cytopathic effect (CPE) inhibition was measured. The results showed that Selenazofurin was markedly inhibitory to influenza A virus and presented greater inhibitory effect than Ribavirin, a known active antiviral compound. Selenazofurin inhibited the cytopathic effect and yield of influenza A/NWS/33 virus with 50% effective dose (ED50) ranges of 0.7 to 1.4 ug/ml. In other experiments, selenazofurin has been reported to have remarkably broad-spectrum antiviral activity. The results of this study indicate that the antiviral activity of the compound extends to the influenza A virus. ¹⁴ Wray S K, Smith R H, Gilbert B E, Knight V. Effects of selenazofurin and ribavirin and their 5′-triphosphates on replicative functions of influenza A and B viruses. Antimicrob Agents Chemother. January 1986;29(1):67-72.

2. Influenza A Virus (Strain A/Wilson-Smith/1933 H1N1), MDCK Cells, Inhibition of Virus Replication

An in vitro experiment¹⁵ showed that selenazofurin, an organoselenium compound, is a potent inhibitor of viral replication in cells infected with influenza A virus. In this experiment, Madin Darby canine kidney (MDCK) cells were infected with 10-50% tissue culture infective doses of influenza virus A/WSN. After an adsorption period of 1 hour, the virus inoculation was removed, and 0.5 ml portions of viral growth medium with or without selenazofurin were added. The selenazofurin concentrations in these studies ranged between 1 to 200M. After 72 hours, virus growth was measured by hem adsorption of 0.05% guinea pig erythrocytes. The results showed that selenazofurin inhibited the growth of influenza A virus. The 50% inhibitory dose (ID50 50% of the wells exhibiting viral growth) of selenazofurin was 25 μM for influenza A virus. At these concentrations, and up to 1 mM, selenazofurin showed no cytotoxic effect on cells. The antiviral potency of selenazofurin in these tests was slightly greater than that of the related compound ribavirin. ¹⁵ Wray S K, Smith R H, Gilbert B E, Knight V. Effects of selenazofurin and ribavirin and their 5′-triphosphates on replicative functions of influenza A and B viruses. Antimicrob Agents Chemother. January 1986;29(1):67-72.

3. Influenza A Virus (Strain A/Bangkok/1/1979 H3N2), BECs, Enhancement of Defense Responses and Decrease in Apoptosis

An in vitro experiment¹⁶ showed that that selenium deficiency significantly impairs the influenza-induced host defense responses in human airway epithelial cells and that adequate selenium levels improve the immune defense. In this experiment, primary human bronchial epithelial cells (BEC) were grown either under selenium-adequate (Se+) or selenium-deficient (Se−) conditions. The cells were incubated with 320 HAU of influenza A Bangkok 1/79 from the apical side for 1 hour. Effects of selenium deficiency on influenza virus infections were assessed 24 hours post infection. The results showed that Se deficiency enhanced influenza-induced apoptosis. The number of apoptotic cells infected with influenza was remarkably greater under the selenium-deficient (Se−) conditions. ¹⁶ Jaspers I, Zhang W, Brighton L E, Carson J L, Styblo M, Beck M A. Selenium deficiency alters epithelial cell morphology and responses to influenza. Free Radic Biol Med. Jun. 15, 2007;42(12):1826-37.

4. Influenza A Virus (Strain A/Bangkok/1/1979 H3N2), Mice, Decrease in Pathology and Inflammatory Response, Improvement of Immune Response

An in vivo experiment¹⁷ showed that selenium deficiency in mice intensifies the influenza virus infection, and causes an increase in pathology and reduces the efficiency of the immune response in comparison to mice with adequate Selenium levels. In this experiment, three weeks old C57B1/6J male mice were fed specified diets, either adequate or deficient in Selenium, for 4 weeks prior to virus inoculation. Mice were than infected with 10 HAU (hemagglutination units) of the influenza A/Bangkok/1/79 virus. The results showed that mice fed the selenium deficient diet had significantly more inflammation at days 4, 6, 10, and 21 post infection than mice fed the selenium adequate diet. The lung pathology in the selenium adequate mice began to diminish after day 6, whereas the selenium deficient mice still had severe pathology even at day 21 post infection. The percentage of CD8+ cells (and, to a lesser extent, CD4+ cells) dropped in the selenium deficient animal when compared with the selenium adequate mice, at day 10 post infection. At all time points, mRNA for IFN gamma and IL-2 (crucial for the immune response against the virus) were much less abundant in the selenium deficient mice than in the selenium adequate mice. ¹⁷ Beck M A, Nelson H K, Shi Q, Van Dael P, Schiffrin E J, Blum S, Barclay D, Levander O A. Selenium deficiency increases the pathology of an influenza virus infection. FASEB J. June 2001;15(8):1481-3.

F. Propolis

Propolis is a resinous mixture that honey bees collect from tree buds, sap flows, or other botanical sources. Propolis is made by integrating the resinous mixture with beeswax and other bee secretions. Propolis chemical composition varies considerably from region to region, along with the vegetation and combines approximately 50 constituents, primarily resins and vegetable balsams (50%), waxes (30%), essential oils (10%), and pollen (5%). In some areas propolis has been documented to contain polyprenylated benzophenones, viscidone, naphthoquinone epoxide, 4-hydroxy-3,5-diprenyl cinnamic acid, sinapic acid, isoferulic acid, caffeic acid and chrysin.

1. Influenza A Virus (Strain A/Puerto Rico/8/1934 H1N1), CAM Tissue Cultures, Inhibitory Effect on Viral Reproduction

An in vitro experiment¹⁸ showed the anti viral activity of esters of substituted cinnamic acids, identical with or analogous to some of the constituents of the Et₂O fraction of propolis. In this experiment, tissue cultures from surviving chorioallantoic membranes (CAM), were exposed to the influenza virus with or with out the substances (propolis fraction analogs) for 48 hours. The inhibitory effect was determined in one-step experiments by the difference of the infectious titers of control and treated viruses. The results showed that the reproduction of influenza A/PR/8 was effectively inhibited by two of the compounds tested (these compounds are identical to those found in propolis). Minimal inhibitory concentrations (MIC) were 50 and 25 μg/ml respectively. ¹⁸ Serkedjieva J, Manolova N, Bankova V. Anti-influenza virus effect of some propolis constituents and their analogues (esters of substituted cinnamic acids). J Nat Prod. March 1992;55(3):294-302.

2. Influenza A Virus (Strain A/Puerto Rico/8/1934 H1N1), Mice, Reduction of the HA Titers and Mortality, Increase in Mean Survival Length

An in vivo experiment¹⁹ showed that the aqueous extract of propolis possesses anti viral effect against influenza virus A/PR8/34 in infected mice. In this experiment, propolis extract administered intranasally to mice 3 hours before virus inoculation or 3 hours after virus inoculation. Propolis anti viral activity was determined by quantitative micro hemagglutination test (HA), extent of mortality and mean survival length. The results showed that administration of propolis extract 3 hours before virus inoculation led to a reduction of the HA titers recorded in the lung suspensions from infected mice, but to no reduction in mortality or increase in mean survival length. When the extract was administered 3 hours after virus inoculation, the reduction in HA titer was accompanied by a slight decrease in mortality and increase in mean survival length. ¹⁹ Esanu V, Prahoveanu E, Crisan I, Cioca A. The effect of an aqueous propolis extract, of rutin and of a rutin-quercetin mixture on experimental influenza virus infection in mice. Virologie. July-September 1981;32(3):213-5.

### V. Preferred Embodiments

The examples showed that GA, quercetin, EGCG, cinnamaldehyde or cinnamic acid, propolis, and selenium, have anti influenza activities. Therefore, a preferred embodiment of the current invention is an effective dose of one of these nutritional supplements, or other nutritional supplements that can serve a source for these nutritional supplement, such as licorice, which can serve as a source of GA, green tea, which can serve as a source of EGCG, cinnamon, which can serve as a source of cinnamaldehyde or cinnamic acid, etc. In addition, a preferred embodiment of the current invention is any combination of some or all of these nutritional supplements, for example, a capsule that include licorice, quercetin, green tea, cinnamon, and selenium.

While the above describes what are presently believed to be the preferred embodiments of the invention, those skilled in the art will realize that changes and modifications may be made thereto without departing from the spirit of the invention. It is intended to claim all such changes and modifications that fall within the true scope of the invention.

What is claimed is:

1. A compound, wherein said compound includes at least three agents selected from the group consisting of licorice, glycyrrhizic acid, quercetin, green tea, epigallocatechin gallate, cinnamon, cinnamaldehyde, cinnamic acid, selenium, and propolis.

2. The method in claim 1, wherein said compound includes at least four of said agents.

3. The method in claim 1, wherein said compound includes at least five of said agents.

4. The method in claim 1, wherein said compound includes at least six of said agents.

5. The method in claim 1, wherein said compound includes licorice, quercetin, green tea, cinnamon, and selenium.

6. The method in claim 1, wherein said compound includes licorice, quercetin, green tea, cinnamon, propolis, and selenium.

7. A method for treating or preventing an infection with an influenza virus in an animal or human subject comprising the administration of a compound to said subject, wherein said compound consists of least two agents selected from the group consisting of licorice, glycyrrhizic acid, quercetin, green tea, epigallocatechin gallate, cinnamon, cinnamaldehyde, cinnamic acid, selenium, and propolis.

8. The method in claim 7, wherein said compound includes at least three of said agents.

9. The method in claim 7, wherein said compound includes at least four of said agents.

10. The method in claim 7, wherein said compound includes at least five of said agents.

11. The method in claim 7, wherein said compound includes licorice, quercetin, green tea, cinnamon, and selenium.

12. The method in claim 7, wherein said compound includes licorice, quercetin, green tea, cinnamon, propolis, and selenium.

13. The method in claim 7, wherein said influenza virus is influenza virus H1N1 .
